(19) United States
(12) Patent Application Publication
(10) Pub. No.: US 2009/0264787 A1
(43) Pub. Date: Oct. 22, 2009

(54) Communication and Device Control System Based on Multi-Frequency, Multi-Phase Encoded visual Evoked Brain Waves

(76) Inventors: Po-lei Lee, Jhongli City (TW); Kuo-kai Shyu, Jhongli City (TW); Chi-hsun Wu, Jhongli City (TW); Jyun-jie Sie, Jhongli City (TW); Ming-huan Lee, Jhongli City (TW)

Correspondence Address: HDLS Patent & Trademark Services, P.O. BOX 220746, CHANTILLY, VA, 20153-0746, US

(21) Appl. No.: 12/179,639

(22) Filed: Jul. 25, 2008

(30) Foreign Application Priority Data
Apr. 16, 2008 (TW) 097113807

(51) Int. Cl. A61B 5/04

(52) U.S. Cl. 600/544

(57) ABSTRACT

In a driving control system actuated by visual evoked brain waves which are induced by a multi-frequency and multi-phase encoder, and a corresponding method configured for analyzing brain wave signals in order to control at least one peripheral equipment, the driving control system includes an optical flash generating device, a brain wave signal measurement device, a signal processing and analyzing device and a control device. The optical flash generating device is configured for generating at least one flash light source by a multi-frequency and multi-phase encoder. The brain wave signal measurement device is configured for measuring a brain wave signal inducing by a user gazing the flash light source. The signal processing and analyzing device is configured for calculating the frequency and the phase of the brain wave by a mathematical method, and analyzing whether the frequency and the phase of the brain wave same to the those of the optical flash generating device. The control device is configured for sending out the frequency and the phase analyzed by the signal processing and analyzing device and same to those of the optical flash generating device so as to control at least one peripheral equipment.

### BACKGROUND

The present invention relates to a driving control system actuated by visual evoked brain waves which are induced by a multi-frequency, multi-phase encoder, and a corresponding method configured for analyzing brain wave signals in order to control at least one peripheral equipment.

### DESCRIPTION OF THE RELATED ART

With the rapid development of the modern technology, humankind can measure physiological signals, such as blood pressure, heart electrical activity, muscle electrical activity or brain electrical activity, by means of those advanced physiological measure technology in effectively, easily and noninvasive ways. By further analyzing and processing the physiological signals, the physiological signals can be utilized as a new interface for the communication between users and external environments. The developments of brain wave recording technology and the neurological science in recent years bring in the maturity of a new technology for users to communicate with external environments using his/her brain waves, independent of peripheral muscle nerve and activities. The technology is called brain computer interface (BCI).

The brain computer interface employs brain signals to make users communicate with external environments directly. The main issues for developing a BCI depend on: (1) designing a proper task to induce particular brain waves; (2) a signal processing step to extract the induced brain wave with high signal-to-noise ratio; (3) a signal processing step to distinguish the expected brain wave from task-unrelated brain waves; (4) one or more external devices controlled by the extracted brain wave.

Conventional human machine interface (HMI) for disabled patients mainly constructed by a voice control input interface or a body control keyboard input interface. However, some users, who are suffering neural or muscular incapability and can not speak as sharpness as normal people, may have problem to operate the periphery equipment. Besides, regarding the body control keyboard input interface, it includes a keyboard and a mouse, both of which are indispensable devices to operate the periphery equipment. The body control keyboard interface may hurt people and induce various health problems, such as neck pain, poor blood cycle, muscle fatigue, etc., by faulty posture and lack of body extending, if a normal user uses the body control keyboard input interface for a long time. Furthermore, the body control keyboard input interface is also not suitable for the users, who are suffering neural or muscular incapability.

The aforementioned HMIs of the periphery equipment generally are only suitable for particular groups of patients who still can use voice or limb movement to operate the periphery equipments. Those HMIs are not suitable for the users, who are suffering neural or muscular incapabilities, having problem with voluntary speech or limb movements.

Accordingly, a system for solving the aforementioned problems is needed.

### BRIEF SUMMARY

A driving control system for visual evoked brain wave, induced by multi-frequency and multi-phase encoder, in accordance with an exemplary embodiment of the present invention is provided. The driving control system is configured for the use of brain wave signals to control at least one periphery equipment. The driving control system includes an optical flash generating device, a brain wave signal measurement device, a signal processing and analyzing device, and a control device. The optical flash generating device is configured for generating at least one flash light source by a multi-frequency and multi-phase encoder. The optical flash generating device includes a programmable chip and at least one light emitting element arranged therein. The programmable chip is configured for generating a multi-channel phase angle delay time by the multifrequency phase encoder, and driving and flashing at least one of the light emitting elements by the multi-channel phase angle delay time. The brain wave signal measurement device is configured for measuring a brain wave signal induced when the user gazing at one of the light emitting elements, and transmitting the brain wave signal to the signal processing and analyzing device. The brain wave signal measurement device includes a brain measurement system, a signal amplifier and an analog-to-digital converter arranged therein. The brain measurement system is configured for measuring the brain wave signals. The amplifier is configured for amplifying the brain wave signal measured by the brain measurement system. The analog-to-digital converter is configured for converting the brain wave signal amplified by the amplifier. The signal processing and analyzing device is configured for calculating a frequency and a phase of the brain wave signal by a mathematical method, and analyzing whether the frequency and the phase of the brain wave same to those of the optical flash generating device. The control device is configured for sending out the frequency and the phase analyzed by the signal processing and analyzing device, and same to those of the optical flash generating device to control at least one of the peripheral equipments.

A method used into a driving control system for visual evoked brain wave by multifrequency phase encoder, in accordance with an exemplary embodiment of the present invention, is provided. The method is configured for the use of brain wave signals to control at least one periphery equipment. The method employing a signal processing and analyzing device of the driving control system to perform following steps:

receiving a multi-channel phase angle delay time generated by a programmable chip with a multifrequency phase encoder, the programmable chip transmitting the multi-channel phase angle delay time to at least one of the light emitting elements for driving and flashing at least one of the light emitting elements;

receiving a brain wave signal sent out from a brain wave signal measurement device, and forming a reference signal based on the brain wave signal when a user gazes at least one of the light emitting elements for the first time;

removing the brain wave signal with no corresponding frequency by a narrow band-pass filter, averaging superposedly the brain wave signal sent out from the brain wave signal measurement device, and comparing the brain wave signal with the reference signal; and

transmitting control commands of the brain wave signal to a control device for controlling at least one periphery equipment, if the brain wave having a frequency and a phase same to those of at least one of the light emitting elements.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a schematic, frame diagram of a driving control system, in accordance with an exemplary embodiment of the present invention;

FIG. 2 is a schematic, sequence chart for generating flash lights by an multi-frequency, multi-phase encoder of the present invention; and

FIG. 3 is a schematic, flowing chart for analyzing the signal of the present invention.

### DETAILED DESCRIPTION

Reference will now be made to the drawings to describe exemplary embodiments of the present driving control system for visual evoked brain wave by multifrequency phase encoder, in detail. The following description is given by way of example, and not limitation.

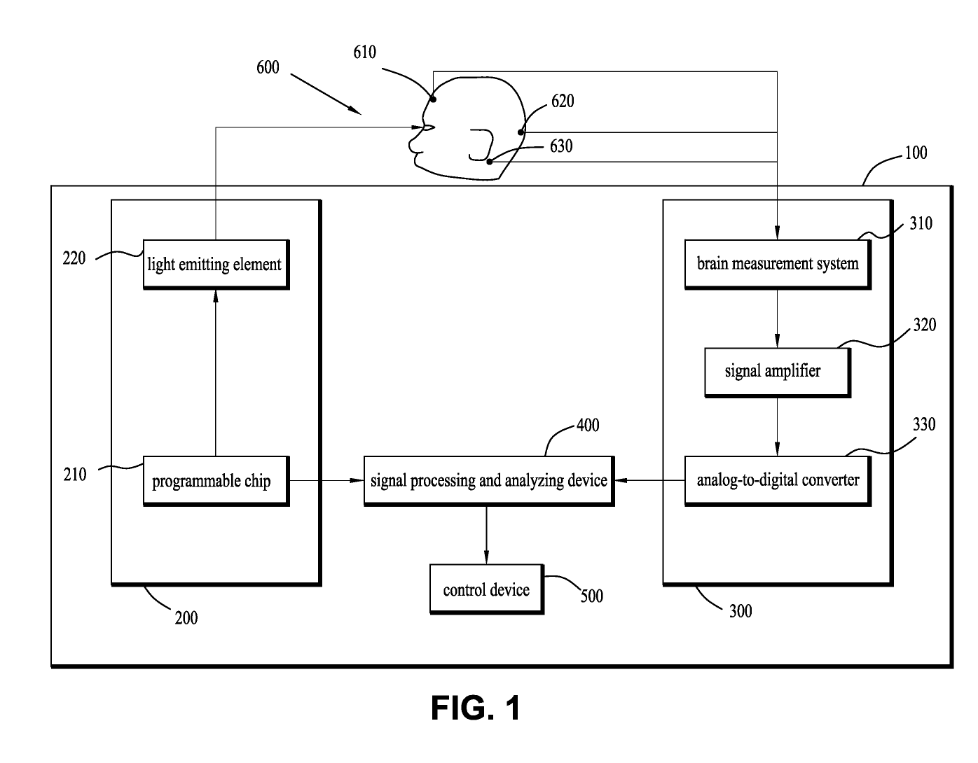

Referring to FIG. 1, a driving control system for visual evoked brain wave by multi-frequency and multi-phase encoder, in accordance with an exemplary embodiment of the present invention, is provided. The driving control system 100 includes an optical flash generating device 200, a brain wave signal measurement device 300, a signal processing and analyzing device 400 and a control device 500. The optical flash generating device 200 is configured for generating at least one flash light source by a multi-frequency and multi-phase encoder. The optical flash generating device 200 includes a programmable chip 210 and at least one light emitting element 220. The programmable chip 210 is selected from one of a group consisting of a field programmable gate array (FPGA), a single chip and a microprocessor. The light emitting element 220 selected from one of a group consisting of a light emitting diode (LED), a flash screen and an element configured for emitting visible light. The programmable chip 210 generates a multi-channel phase angle delay time by the multi-frequency and multi-phase encoder, and drives the light emitting element 220 by the multi-channel phase angle delay time, such that the light emitting element 220 flashes. The brain wave signal measurement device 300 is configured for measuring a brain wave signal induced by a user gazing the light emitting element 220, and transmitting the brain wave signal to the signal processing and analyzing device 400. The brain wave signal measurement device 300 includes a brain measurement system 310, a signal amplifier 310 and an analog-to-digital converter 330 arranged therein. The brain measurement system 310 may be one of 10-20 type systems designed by the International Brain Wave Association. The brain measurement system 310 employs at least one positive electrode chip attached on a brain optical zone (OZ) 620, a negative electrode chip attached on a postauricular mastoid 630, and a grounding electrode chip attached on a forehead 610, to measure the brain wave signal. The measured brain wave signal is amplified by the signal amplifier 320, and then converted from an analog signal to a digital signal by the analog-to-digital converter 330.

The signal processing and analyzing device 400 is configured for calculating the frequency and the phase of the brain wave signal by a mathematical method, and then analyzing whether the frequency and the phase of the brain wave signal same to those of the optical flash generating device 200 for improving signal-to-noise ratio. The mathematical method is one of a group consisting of the Fourier transform method, the temporal ensemble averaging method, the wavelet method, and a method for analyzing a phase of a sine wave. The control device 500 is configured for sending out the frequency and the phase analyzed by the signal processing and analyzing device 400 and same to those of the optical flash generating device 200, to control at least one periphery equipment.

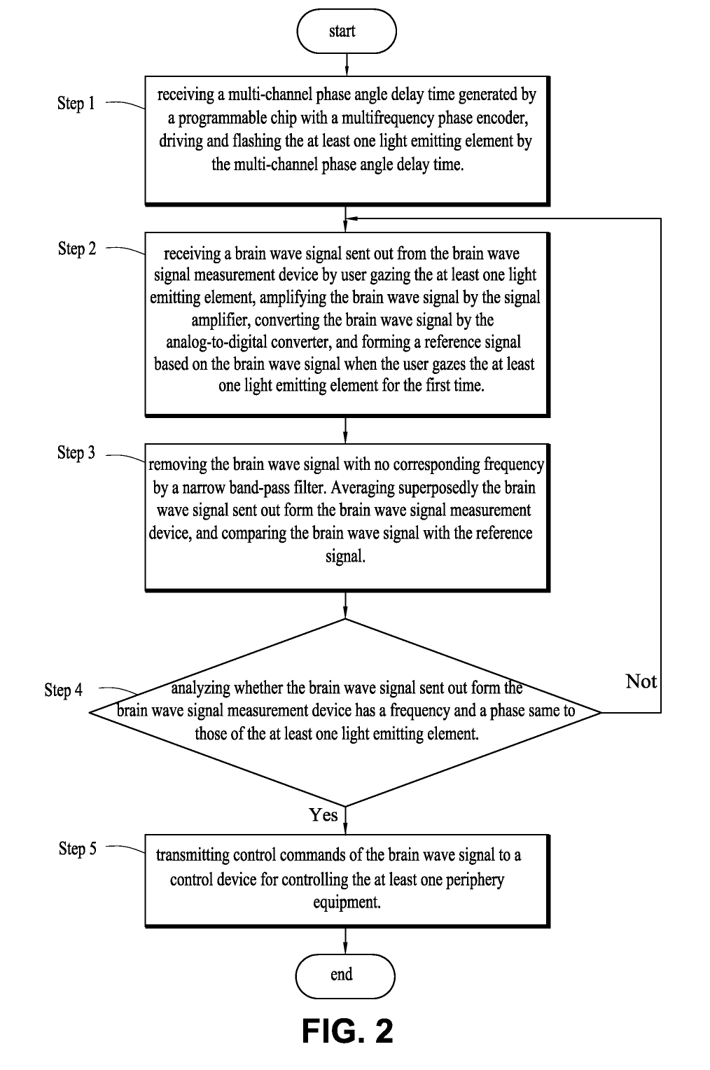

Referring to FIG. 2, the schematic, sequence chart of the flash light source generated by the optical flash generating device 200 with the multi-frequency and multi-phase encoder, is provided. The programmable chip 210 employs an operational formula

$$θ n = \frac{2 π}{x} \times ( n - 1 )$$

to form a phase code, wherein θₙ is a phase angle of a channel n; n is a serial number of a flash channel; and x is an amount of the at least one light emitting element. The operational formula

$$θ n = \frac{2 π}{x} \times ( n - 1 )$$

is processed and transformed into a transforming formula with time to phase,

$${t}_{n} = \frac{{θ}_{n}}{{ω}_{m}} = \frac{1}{2 π {f}_{m}} \times \frac{2 π ( n - 1 )}{x} = \frac{{t}_{m}}{x} \times ( n - 1 )$$

by an equation θ=ωt, wherein tₙ is a delay time of the channel n; tₘ is the channel flash cycle (the inversion of the flash frequency); and fₘ is the channel flash frequency. If a first channel flash frequency (f1) is respectively equal to 1, 2, 3, and 4 and inserted into the transforming formula

$${t}_{n} = \frac{{θ}_{n}}{{ω}_{m}} = \frac{1}{2 π {f}_{m}} \times \frac{2 π ( n - 1 )}{x} = \frac{{t}_{m}}{x} \times ( n - 1 ) ,$$

four channel phase angles (θ₁, θ₂, θ₃, θ₄) are achieved, and the delay time (t₁˜t₄) of the four channels ‘1’˜‘4’ are achieved. The light emitting element 220 is driven according to the sequence of the delay time of the four channels, to generate four flash light sources. Then, making a second channel flash frequency (f2) equal to 5, 6, 7, 8, and inserted into the transforming formula

$${t}_{n} = \frac{{θ}_{n}}{{ω}_{m}} = \frac{1}{2 π {f}_{m}} \times \frac{2 π ( n - 1 )}{x} = \frac{{t}_{m}}{x} \times ( n - 1 ) ,$$

such that another four flash light sources are generated again by using the above method. Thus, eight flash light sources are generated.

A sine wave formula S(t)=sin(ωt+θ) is used to prove that the phase code has the phase angle (θ) by the time delay. If the phase angle (θ) of the sine wave formula S(t)=sin(ωt+θ) is equal to a product of an angle speed (ω) and a time constant (t′), the sine wave formula may be transformed into S(t)=sin(ωt+ωt′)=sin(ω(t+t′), and then achieve s( [symbol] −t′)=sin(ω [symbol] ) through an variable transform [symbol] =t+t′. From the above, it is known that the sine wave formula S(t)=sin(ωt+θ) may generate a sine wave function having the phase angle (θ) by the time delay. That is, the flash sequence phase angle (θ) needed in the present invention is achieved by the time delay.

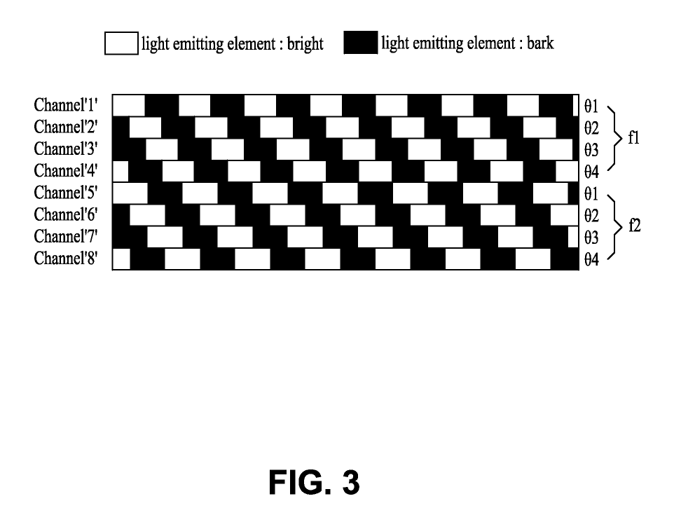

Referring to FIG. 3, when the driving control system 100 is used to measure the brain wave signal of a user, and analyze the relation between the brain wave signal and the flash light source to operate the periphery equipment, the signal processing and analyzing device 400 of the driving control system 100 performs following steps.

Step 1 is for receiving the multi-channel phase angle delay time generated by the programmable chip 210 with the multi-frequency and multi-phase encoder. At the same time, the programmable chip 210 transmits the multi-channel phase angle delay time to the at least one light emitting element 220 selected from one of a group consisting of a light emitting diode, a flash screen and an element configured for emitting visible light, such that the at least one light emitting element 220 flashes by the multi-channel phase angle delay time.

Step 2 is for receiving the brain wave signal sent out from the brain wave signal measurement device 300. The brain wave signal is generated by using the brain measurement system 310 to detect the brain visual cortex area 620 of the user, which gazes the at least one light emitting element 220. The brain wave signal is then amplified by the signal amplifier 320, and converted from an analog signal to a digital signal by the analog-to-digital converter 330. A reference signal is formed by measuring the brain wave signal when the user gazes the at least one light emitting element 220 for the first time.

Step 3 is for removing the brain wave signal with no corresponding frequency by a narrow band-pass filter. The brain wave signal sent out from the brain wave signal measurement device 300 is averaged superposedly, and then compared with the reference signal.

Step 4 is for analyzing whether the frequency and the phase of the brain wave signal sent out from the brain wave signal measurement device 300 same to those of the light emitting element 220, if yes, performing step 5; if not, turning back to perform step 2.

Step 5 is for transmitting control commands of the brain wave signal to the control device 500 for controlling the periphery equipment, such that the processing steps by using the driving control system 100 to control the periphery equipment are over.

From the above, the present invention has many advantages as follows.

Firstly, since the present invention employs a steady-state visual evoked potential system with frequency encoding cooperated with a random flash visual evoked potential system, the present invention may achieve the multi-channel with less flash frequency, and have many advantages, such as visual display with flash sequence in series, strong anti-interfering capability for other physiological signal, steady and quick analyzing time, and less measuring electrodes, etc. Thus the present invention is novel and unobvious.

Furthermore, the driving control system 100 and the corresponding method of the present invention, may not need periphery neuron and muscle, and just use the brain wave signal to control the periphery equipment for communicating with out, transmitting information, auto-motion and self-care, etc. Thus the present invention may improve the life quality of people, and is practicable.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A driving control system actuated by visual evoked brain waves which are induced by a multi-frequency and multi-phase encoder, the driving control system being configured for use of brain wave signals to control at least one periphery equipment, the driving control system comprising:
an optical flash generating device configured for generating at least one flash light source by a multi-frequency, multi-phase encoder, the optical flash generating device including a programmable chip and at least one visible light emitting element arranged therein, the programmable chip being configured to generate multi-frequency, multi-phase time sequence for a multi-channel flash emitter, and driving and flashing the at least one visible light emitting element by a multi-channel phase angle delay time;
a brain wave signal measurement device configured for measuring a brain wave signal induced by a user gazing at the multi-channel light emitting element, the brain wave signal measurement device including a brain measurement system, a signal amplifier and an analog-to-digital converter arranged therein, the brain measurement system being configured for measuring the brain wave signal, the amplifier being configured for amplifying the brain wave signal measured by the brain measurement system, and the analog-to-digital converter being configured for digitizing the brain wave signal amplified by the amplifier;
a signal processing and analyzing device configured for receiving the brain wave signal from the brain wave signal measurement device, calculating frequency and phase of the brain wave signal by mathematical method, and analyzing whether the frequency and the phase of the brain wave match to those of the optical flash generating device; and
a control device configured for sending out commands according to an analyzed result made by the signal processing and analyzing device, that the frequency and the phase matching to those of the optical flash generating device, to control the at least one peripheral equipment.

2. The driving control system as claimed in claim 1, wherein the programmable chip employs an operational formula
$$θ n = \frac{2 π}{x} \times ( n - 1 )$$
to form a phase code, θₙ is a phase angle of a channel n; n is a serial number of a flash channel; x is an amount of the at least one light emitting element; the operational formula
$$θ n = \frac{2 π}{x} \times ( n - 1 )$$
is processed and transformed into a transforming formula with time to phase
$${t}_{n} = \frac{{θ}_{n}}{{ω}_{m}} = \frac{1}{2 π {f}_{m}} \times \frac{2 π ( n - 1 )}{x} = \frac{{t}_{m}}{x} \times ( n - 1 )$$
by an equation θ=ωt, tₙ is a delay time of the channel n; tₘ is a channel flash cycle; and fₘ is a channel flash frequency.

3. The driving control system as claimed in claim 1, wherein the multi-channel phase angle delay time includes one of a signal channel flash frequency and a combination of combining at least two channel flash frequencies.

4. The driving control system as claimed in claim 1, wherein the programmable chip is one of a group consisting of a field programmable gate array (FPGA), a single chip and a microprocessor.

5. The driving control system as claimed in claim 1, wherein the at least one light emitting element is one of a group consisting of a light emitting diode, a flash screen and an element configured for emitting visible light.

6. The driving control system as claimed in claim 1, wherein the brain measurement system is to measure ongoing brain waves, to be detected by an electrode contact removably attached to the surface of the head, the electrodes are generally connected by a conductive paste and the system of attaching the electrodes generally follows the International Association 10-20 system which specifies anatomical points to which the various electrodes should be connected, and the electrodes are located on visual cortex area, postauricular mastoid as reference, and grounded on forehead.

7. The driving control system as claimed in claim 1, wherein the mathematical method is one of a group consisting of the Fourier transform method, the temporal ensemble averaging method, the wavelet method, and a method configured for analyzing a phase of a sine wave.

8. A method used into a driving control system for visual evoked brain wave by multi-frequency and multi-phase encoder, the method being configured for using brain wave signals to control at least one periphery equipment, the method employing a signal processing and analyzing device of the driving control system to perform following steps:
receiving a multi-channel phase angle delay time generated by a programmable chip with a multi-frequency and multi-phase encoder, the programmable chip transmitting the multi-channel phase angle delay time to at least one light emitting element for driving and flashing the at least one light emitting element;
receiving brain wave signal sent out from the brain wave signal measurement device, and forming a signal waveform as template based on the brain wave signal when a user initially gazes at the at least one light emitting element;
removing the brain wave signal with no corresponding frequency by a narrow band-pass filter, averaging superposedly the brain wave signal sent out from the brain wave signal measurement device, and comparing the brain wave signal with the template signal; and
transmitting control commands of the brain wave signal to a control device for controlling the at least one periphery equipment, if the brain wave having both the same frequency and phase matched to those of the at least one light emitting element.

9. The method as claimed in claim 8, wherein the programmable chip employs an operational formula
$$θ n = \frac{2 π}{x} \times ( n - 1 )$$
to form a phase code, θₙ is a phase angle of a channel n; n is a serial number of a flash channel; X is an amount of the at least one light emitting element; the operational formula
$$θ n = \frac{2 π}{x} \times ( n - 1 )$$
is processed and transformed into a transforming formula with time to phase
$${t}_{n} = \frac{{θ}_{n}}{{ω}_{m}} = \frac{1}{2 π {f}_{m}} \times \frac{2 π ( n - 1 )}{x} = \frac{{t}_{m}}{x} \times ( n - 1 )$$
by an equation θ=ωt, tₙ is a delay time of the channel n; tₘ is a channel flash cycle; and fₘ is a channel flash frequency.

10. The method as claimed in claim 8, wherein the multi-channel phase angle delay time includes one of a signal channel flash frequency and a combination of combining at least two channel flash frequencies.

11. The method as claimed in claim 8, wherein the brain wave signal sent out from the brain wave signal measurement device, is measured from a brain visual cortex area of a user, which gazes at the at least one light emitting element, by a brain measurement system, the measured brain wave signal is then amplified by a amplifier and digitized from an analog signal to a digital signal by an analog-to-signal converter.

12. The method as claimed in claim 8, wherein the programmable chip is one of a group consisting of a field programmable gate array, a single chip and/or a microprocessor.

13. The method as claimed in claim 8, wherein the at least one light emitting element is one of a group consisting of a light emitting diode, a flash screen and an element configured for emitting visible light.

14. The method as claimed in claim 11, wherein the brain measurement system is one of 10-20 type systems designed by the International Brain Wave Association, the brain measurement system includes at least one positive electrode chip attached on a brain optical zone, a negative electrode chip attached on a postauricular mastoid, and a grounding electrode chip attached on a forehead to measure the brain wave signal.
